(19) United States
(12) Patent Application Publication
(10) Pub. No.: US 2003/0032661 A1
(43) Pub. Date: Feb. 13, 2003

(54) Pramipexole as an anticonvulsant

(75) Inventor: Jens Croenlein, Mittelbiberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma KG, Ingelheim (DE)

(21) Appl. No.: 10/197,734

(22) Filed: Jul. 18, 2002

Related U.S. Application Data
(60) Provisional application No. 60/312,216, filed on Aug. 14, 2001.

(30) Foreign Application Priority Data
Aug. 2, 2001 (DE) 101 37 453.4

(51) Int. Cl. A61K 31/428

(52) U.S. Cl. 514/367

(57) ABSTRACT

A method for providing anticonvulsant treatment in a patient in need thereof, the method comprising administering to the patient an effective amount of 2-amino-4,5,6,7-tetrahydro-6-n-propylaminobenzothiazole or a pharmacologically acceptable acid addition salt, hydrate, or solvate thereof.

### RELATED APPLICATIONS

Benefit under 35 U.S.C. § 119(e) of prior provisional application Serial No. 60/312,216, filed Aug. 14, 2001, is hereby claimed.

### FIELD OF THE INVENTION

The invention relates to the use of 2-amino-4,5,6,7-tetrahydro-6-n-propylaminobenzothiazole, the (+) or (−) enantiomer thereof, the pharmacologically acceptable acid addition salts thereof, as well as hydrates and solvates, for preparing a pharmaceutical composition with an anticonvulsant activity.

### BACKGROUND OF THE INVENTION

2-Amino-6-n-propylamino-4,5,6,7-tetrahydrobenzothiazole is a dopamine agonist which is also known in the art by the name pramipexole. Pramipexole and processes for preparing it are known for example from EP-A-186 087 and U.S. Pat. No. 4,886,812. It is known in particular for the treatment of schizophrenia and particularly for the treatment of Parkinson's disease. The prior art further discloses, for example, that pramipexole lowers the prolactin serum level (DE 38 43 227).

### DESCRIPTION OF THE INVENTION

Surprisingly, it has been found that pramipexole can be used in therapeutically effective doses as an anticonvulsant for treating cerebral seizures.

Accordingly, the present invention relates to the use of 2-amino-4,5,6,7-tetrahydro-6-n-propylaminobenzothiazole, optionally in the form of its (+) or (−) enantiomer, optionally in the form of the pharmacologically acceptable acid addition salts, as well as hydrates and solvates, for preparing a pharmaceutical composition with an anticonvulsant activity.

The present invention further relates to the use of 2-amino-4,5,6,7-tetrahydro-6-n-propylaminobenzothiazole, optionally in the form of the (+) or (−) enantiomer thereof, optionally in the form of the pharmacologically acceptable acid addition salts thereof, as well as hydrates and solvates, for preparing a pharmaceutical composition for the prevention and/or treatment of cerebral seizures.

Within the scope of the present invention, the term cerebral seizures means the same as cerebral convulsions.

Moreover, the present invention relates to the use of 2-amino-4,5,6,7-tetrahydro-6-n-propylaminobenzothiazole, optionally in the form of the (+) or (−) enantiomer thereof, optionally in the form of the pharmacologically acceptable acid addition salts thereof, as well as hydrates and solvates, for preparing a pharmaceutical composition for the prevention and/or treatment of generalized seizures (absences, also atypical absences, myoclonic, clonic, tonic, and tonic-clonic seizures), focal (simple and complex focal) and secondary generalized seizures.

In particular, the present invention relates to the use of 2-amino-4,5,6,7-tetrahydro-6-n-propylaminobenzothiazole, optionally in the form of the (+) or (−) enantiomer thereof, optionally in the form of the pharmacologically acceptable acid addition salts thereof, as well as hydrates and solvates, for preparing a pharmaceutical composition for the prevention and/or treatment of epilepsy.

For treatment and/or prevention of the medical indications described above, in addition to monotherapy using pramipexole it is also possible, as an alternative, to carry out a combined therapy using pramipexole with one or more, preferably one other pharmaceutically active compound. A combination with other anticonvulsants may prove particularly effective, for example.

Pramipexole may be used to treat the abovementioned conditions in conjunction, for example, with one or more, preferably one of the following substances: carbamazepine, oxcarbamazepine, valproic acid, diphenylhydantoin, ethosuximide, mesuximide, phenobarbital, primidone, benzodiazepines (preferably diazepam, clonazepam or clobazam), corticotrophin, corticoids, bromides (such as potassium bromide), sultiam, acetazolamide, felbamate, gabapentin, lamotrigine, topiramate, vigabatrin, levetiracetam, and zonisamide.

Pramipexole may be used within the scope of the present invention as a racemate or in the form of its (+) or (−) enantiomer. Moreover, pramipexole may be used in the form of the pharmaceutically acceptable acid addition salts thereof as well as optionally in the form of its hydrates and/or solvates. By pharmaceutically acceptable acid addition salts are meant according to the invention the salts selected from the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid, of which the salts of hydrochloric acid, hydrobromic acid, sulfuric acid, phosphoric acid, and acetic acid are particularly preferred. The salts of hydrochloric acid are particularly important. Most preferably, within the scope of the present invention, therefore, the hydrochlorides of pramipexole are used, pramipexole dihydrochloride being of particular significance. Of the hydrates of pramipexole, pramipexole dihydrochloride monohydrate is particularly preferred.

The dosage of pramipexole naturally depends to a great extent on the clinical picture. For example, without restricting the present invention thereto, pramipexole may be used in doses of about 0.05 mg to 7.5 mg, preferably 0.1 mg to 5 mg per day. These doses are based on pramipexole in the form of its free base. Based on the salt form pramipexole dihydrochloride monohydrate which is preferably used, the doses mentioned above correspond to about 0.07 mg to 10.65 mg, preferably 0.14 mg to 7.1 mg of pramipexole dihydrochloride monohydrate per day.

One possible dosing method, which is to be understood as being merely an illustrative example, is described below, based on pramipexole in the form of its free base: individual dosage titration at weekly intervals depending on activity and acceptability.

1st week: 1 tablet containing 0.088 mg of pramipexole 3 times a day;

2nd week: 1 tablet containing 0.18 mg of pramipexole 3 times a day; and

3rd week and thereafter: ½ tablet containing 0.7 mg of pramipexole 3 times a day. Depending on the severity of the clinical picture, it may sometimes be necessary to administer higher doses of pramipexole. In such cases, maximum doses of about 5 mg to 7.5 mg of pramipexole per day are appropriate.

Within the scope of the use according to the invention pramipexole may be administered orally, transdermally, intrathecally, by inhalation or parenterally. Suitable preparations include, for example, tablets, capsules, suppositories, solutions, syrups, emulsions, dispersible powders, or patches. Regarding possible embodiments of a transdermal preparation which may be used according to the invention, we now refer to the embodiments described by way of example in U.S. Pat. No. 5,112,842, which is hereby incorporated by reference in its entirety. Suitable tablets may be produced, for example, by mixing the active substance or substances with known excipients, for example, inert diluents, such as calcium carbonate, calcium phosphate, or lactose, disintegrants such as corn starch or alginic acid, binders such as starch or gelatine, lubricants such as magnesium stearate or talc, and/or agents for achieving delayed release such as carboxymethylcellulose, cellulose acetate phthalate, or polyvinyl acetate. The tablets may also consist of several layers.

The following are some examples of pharmaceutical preparations which may be used according to the invention. These are intended solely as an illustration without restricting the subject matter of the invention thereto.

| | |
|---|---|
| Ingredient | Amount (mg) |

Tablet 1

| | |
|---|---|
| pramipexole dihydrochloride monohydrate | 1.00 |
| mannitol | 121.50 |
| maize starch | 79.85 |
| highly dispersed silicon dioxide, anhydrous | 2.30 |
| Polyvidone K25 | 2.35 |
| magnesium stearate | 3.00 |
| Total | 210.00 |

Tablet 2

| | |
|---|---|
| pramipexole | 0.5 |
| mannitol | 122.0 |
| maize starch, dried | 61.8 |
| maize starch | 18.0 |
| highly dispersed silicon dioxide, anhydrous | 2.4 |
| Polyvidone K25 | 2.3 |
| magnesium stearate | 3.0 |
| Total | 210.0 |

Tablet 3

| | |
|---|---|
| pramipexole | 0.25 |
| mannitol | 61.0 |
| maize starch | 39.90 |
| highly dispersed silicon dioxide, anhydrous | 1.20 |
| Polyvidone K25 | 1.15 |
| magnesium stearate | 1.5 |
| Total | 105.00 |

Tablet 4

| pramipexole | 0.125 |
|---|---|
| mannitol | 49.455 |
| maize starch, dried | 25.010 |
| maize starch | 7.300 |
| highly dispersed silicon dioxide, anhydrous | 0.940 |
| Polyvidone K25 | 0.940 |
| magnesium stearate | 1.230 |
| Total | 85.000 |
| Ingredient | Amount |

Formulation Suitable for Injection

| | | |
|---|---|---|
| pramipexole dihydrochloride monohydrate | 0.3 | mg |
| sodium chloride | 0.8 | mg |
| benzalkonium chloride | 0.01 | mg |
| water for injection | ad 100 | mL |

We claim:

1. A method for providing anticonvulsant treatment in a patient in need thereof, the method comprising administering to the patient an effective amount of 2-amino-4,5,6,7-tetrahydro-6-n-propylaminobenzothiazole or a pharmacologically acceptable acid addition salt, hydrate, or solvate thereof.

2. The method of claim 1, wherein the 2-amino-4,5,6,7-tetrahydro-6-n-propylaminobenzothiazole is the (+) or (−) enantiomer thereof.

3. A method for prevention and/or treatment of cerebral seizures in a patient in need thereof, the method comprising administering to the patient an effective amount of 2-amino-4,5,6,7-tetrahydro-6-n-propylaminobenzothiazole or a pharmacologically acceptable acid addition salt, hydrate, or solvate thereof.

4. The method of claim 3, wherein the 2-amino-4,5,6,7-tetrahydro-6-n-propylaminobenzothiazole is the (+) or (−) enantiomer thereof.

5. A method for prevention and/or treatment of generalized seizures including absences, atypical absences, and myoclonic, clonic, tonic, and tonic-clonic seizures; simple and complex focal seizures; or secondary generalized seizures in a patient in need thereof, the method comprising administering to the patient an effective amount of 2-amino-4,5,6,7-tetrahydro-6-n-propylaminobenzothiazole or a pharmacologically acceptable acid addition salt, hydrate, or solvate thereof.

6. The method of claim 5, wherein the 2-amino-4,5,6,7-tetrahydro-6-n-propylaminobenzothiazole is the (+) or (−) enantiomer thereof.

7. A method for prevention and/or treatment of epilepsy in a patient in need thereof, the method comprising administering to the patient an effective amount of 2-amino-4,5,6,7-tetrahydro-6-n-propylaminobenzothiazole or a pharmacologically acceptable acid addition salt, hydrate, or solvate thereof.

8. The method of claim 7, wherein the 2-amino-4,5,6,7-tetrahydro-6-n-propylaminobenzothiazole is the (+) or (−) enantiomer thereof.

9. A method for providing anticonvulsant treatment in a patient in need thereof, the method comprising administering to the patient an effective amount of:
(a) 2-amino-4,5,6,7-tetrahydro-6-n-propylaminobenzothiazole or a pharmacologically acceptable acid addition salt, hydrate, or solvate thereof; and
(b) a second pharmaceutical substance.

10. The method of claim 9, wherein the 2-amino-4,5,6,7-tetrahydro-6-n-propylaminobenzothiazole is the (+) or (−) enantiomer thereof.

11. The method of claim 10, wherein the second pharmaceutical substance is an anticonvulsant.

12. The method of claim 10, wherein the second pharmaceutical substance is selected from carbamazepine, oxcarbamazepine, valproic acid, diphenylhydantoin, ethosuximide, mesuximide, phenobarbital, primidone, benzodiazepines, corticotrophin, corticoids, bromides, sultiam, acetazolamide, felbamate, gabapentin, lamotrigine, topiramate, vigabatrin, levetiracetam, and zonisamide.

13. A method for prevention and/or treatment of cerebral seizures in a patient in need thereof, the method comprising administering to the patient an effective amount of:
(a) 2-amino-4,5,6,7-tetrahydro-6-n-propylaminobenzothiazole or a pharmacologically acceptable acid addition salt, hydrate, or solvate thereof, and
(b) a second pharmaceutical substance.

14. The method of claim 13, wherein the 2-amino-4,5,6,7-tetrahydro-6-n-propylaminobenzothiazole is the (+) or (−) enantiomer thereof.

15. The method of claim 14, wherein the second pharmaceutical substance is an anticonvulsant.

16. The method of claim 14, wherein the second pharmaceutical substance is selected from carbamazepine, oxcarbamazepine, valproic acid, diphenylhydantoin, ethosuximide, mesuximide, phenobarbital, primidone, benzodiazepines, corticotrophin, corticoids, bromides, sultiam, acetazolamide, felbamate, gabapentin, lamotrigine, topiramate, vigabatrin, levetiracetam, and zonisamide.

17. A method for prevention and/or treatment of generalized seizures including absences, atypical absences, and myoclonic, clonic, tonic, and tonic-clonic seizures; simple and complex focal seizures; or secondary generalized seizures in a patient in need thereof, the method comprising administering to the patient an effective amount of:
(a) 2-amino-4,5,6,7-tetrahydro-6-n-propylaminobenzothiazole or a pharmacologically acceptable acid addition salt, hydrate, or solvate thereof; and
(b) a second pharmaceutical substance.

18. The method of claim 17, wherein the 2-amino-4,5,6,7-tetrahydro-6-n-propylaminobenzothiazole is the (+) or (−) enantiomer thereof.

19. The method of claim 18, wherein the second pharmaceutical substance is an anticonvulsant.

20. The method of claim 18, wherein the second pharmaceutical substance is selected from carbamazepine, oxcarbamazepine, valproic acid, diphenylhydantoin, ethosuximide, mesuximide, phenobarbital, primidone, benzodiazepines, corticotrophin, corticoids, bromides, sultiam, acetazolamide, felbamate, gabapentin, lamotrigine, topiramate, vigabatrin, levetiracetam, and zonisamide.

21. A method for prevention and/or treatment of epilepsy in a patient in need thereof, the method comprising administering to the patient an effective amount of:
(a) 2-amino-4,5,6,7-tetrahydro-6-n-propylaminobenzothiazole or a pharmacologically acceptable acid addition salt, hydrate, or solvate thereof; and
(b) a second pharmaceutical substance.

22. The method of claim 21, wherein the 2-amino-4,5,6,7-tetrahydro-6-n-propylaminobenzothiazole is the (+) or (−) enantiomer thereof.

23. The method of claim 22, wherein the second pharmaceutical substance is an anticonvulsant.

24. The method of claim 22, wherein the second pharmaceutical substance is selected from carbamazepine, oxcarbamazepine, valproic acid, diphenylhydantoin, ethosuximide, mesuximide, phenobarbital, primidone, benzodiazepines, corticotrophin, corticoids, bromides, sultiam, acetazolamide, felbamate, gabapentin, lamotrigine, topiramate, vigabatrin, levetiracetam, and zonisamide.

25. The method according to claim 1, wherein the 2-amino-4,5,6,7-tetrahydro-6-n-propylaminobenzothiazole is used in the form of a pharmacologically acceptable acid addition salt selected from the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

26. The method according to claim 1, wherein the 2-amino-4,5,6,7-tetrahydro-6-n-propylaminobenzothiazole is used in a dose of about 0.05 mg to 7.5 mg per day.

27. The method according to claim 1, wherein the 2-amino-4,5,6,7-tetrahydro-6-n-propylaminobenzothiazole is used in a dose of about 0.1 mg to 5.0 mg per day.

28. The method according to claim 3, wherein the 2-amino-4,5,6,7-tetrahydro-6-n-propylaminobenzothiazole is used in the form of a pharmacologically acceptable acid addition salt selected from the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

29. The method according to claim 3, wherein the 2-amino-4,5,6,7-tetrahydro-6-n-propylaminobenzothiazole is used in a dose of about 0.05 mg to 7.5 mg per day.

30. The method according to claim 3, wherein the 2-amino-4,5,6,7-tetrahydro-6-n-propylaminobenzothiazole is used in a dose of about 0.1 mg to 5.0 mg per day.

31. The method according to claim 5, wherein the 2-amino-4,5,6,7-tetrahydro-6-n-propylaminobenzothiazole is used in the form of a pharmacologically acceptable acid addition salt selected from the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

32. The method according to claim 5, wherein the 2-amino-4,5,6,7-tetrahydro-6-n-propylaminobenzothiazole is used in a dose of about 0.05 mg to 7.5 mg per day.

33. The method according to claim 5, wherein the 2-amino-4,5,6,7-tetrahydro-6-n-propylaminobenzothiazole is used in a dose of about 0.1 mg to 5.0 mg per day.

34. The method according to claim 7, wherein the 2-amino-4,5,6,7-tetrahydro-6-n-propylaminobenzothiazole is used in the form of a pharmacologically acceptable acid addition salt selected from the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

35. The method according to claim 7, wherein the 2-amino-4,5,6,7-tetrahydro-6-n-propylaminobenzothiazole is used in a dose of about 0.05 mg to 7.5 mg per day.

36. The method according to claim 7, wherein the 2-amino-4,5,6,7-tetrahydro-6-n-propylaminobenzothiazole is used in a dose of about 0.1 mg to 5.0 mg per day.
